(19) United States
(12) Patent Application Publication
(10) Pub. No.: US 2017/0219509 A1
(43) Pub. Date: Aug. 3, 2017

(54) Determining Electrophysiological Electrode Quality

(71) Applicant: Draeger Medical Systems, Inc., Andover, MA (US)

(72) Inventors: Pericles Nicholas Bakalos, Maynard, MA (US); Ronald D. Gatzke, Lexington, MA (US)

(21) Appl. No.: 15/014,532

(22) Filed: Feb. 3, 2016

(51) Int. Cl. G01N 27/20; G01N 27/24; A61B 5/0408

(52) U.S. Cl. CPC G01N 27/20; A61B 5/0408; G01N 27/24; A61B 2560/0266

(57) ABSTRACT

Systems and methods are provided for simultaneously determining impedances of a plurality of electrophysiological electrodes. Signals are injected into a first electrophysiological electrode and a second electrophysiological electrode, the injected signals differing in at least one of magnitude and phase. A magnitude and phase of an output of a differential amplifier are evaluated, where the differential amplifier is responsive to outputs of the first electrophysiological electrode and the second electrophysiological electrode. An impedance of the first electrophysiological electrode and an impedance of the second electrophysiological electrode are determined based on the magnitude and the phase of the differential amplifier output.

### FIELD

This disclosure is related generally to electronics fault detection and more particularly to detection of electrophysiological electrode quality, such as an electrode utilized in a biomedical application.

### BACKGROUND

Electrical conductors, such as electrical leads or electrodes, are often used in the acquisition and transmission of electrophysiological signals. Such signals are typically transmitted to a remote location, where the signals are stored and processed to produce useful output. For example, in an electrocardiogram system (an ECG or EKG system) a number of electrodes are placed at different positions on a human body to measure changes in electric potential across different parts of the body. Those changes in electric potential are caused by stimulus, such as the beating of the heart or respiration. Over time and usage, electrodes can age, dry out, or otherwise deteriorate, which can compromise their ability to acquire and transmit signals. For example, as ECG electrodes, often consisting of a conducting gel embedded in the middle of a self-adhesive pad, age and dry out, they become poor transducers for conversion of ionic body currents to electronic currents. As an electrode degrades, its impedance increases, and ECG signal distortion and noise increase, while transduction sensitivity correspondingly decreases. Such electrode deterioration can cause faults in signal acquisition, where deteriorated electrodes can result in limited signal capture or complete signal loss.

### SUMMARY

Systems and methods are provided for simultaneously determining impedances of a plurality of electrodes. Signals are injected into a first electrode and a second electrode, the injected signals differing in at least one of magnitude and phase. A magnitude and phase of an output of a differential amplifier are evaluated, where the differential amplifier is responsive to outputs of the first electrode and the second electrode. An impedance of the first electrode and an impedance of the second electrode are determined based on the magnitude and the phase of the differential amplifier output.

As another example, a system for simultaneously determining impedances of a plurality of electrodes includes a current source configured to inject signals into a first electrode and a second electrode, the injected signals differing in at least one of magnitude and phase. A differential amplifier is configured to receive an output of the first electrode and an output of the second electrode, the differential amplifier being further configured to output a difference signal. A data processor is configured to determine an impedance of the first electrode and an impedance of the second electrode based on a magnitude and phase of the difference signal.

As a further example, an electrocardiogram machine is configured to determine impedances of a plurality of electrodes connected to the electrocardiogram machine, signals that differ in at least one of magnitude and phase being injected into a first electrode and a second electrode. The electrocardiogram machine includes a differential amplifier configured to receive an output of the first electrode and an output of the second electrode, the differential amplifier being further configured to output a difference signal. A data processor is configured to determine an impedance of the first electrode and an impedance of the second electrode based on a magnitude and phase of the difference signal.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 is a block diagram depicting a system for simultaneously determining the quality of a plurality of electrodes.

FIGS. 2A and 2B are a diagram depicting exemplary components of a system for determining qualities of a plurality of electrodes simultaneously.

FIG. 3A is a diagram depicting an example current source for injecting unbalanced currents (magnitude and phase) into a pair of electrodes.

FIG. 3B is a diagram depicting another example current source for injecting unbalanced currents (phase) into a pair of electrodes.

FIG. 3C is a diagram depicting a further example current source for injecting unbalanced currents (magnitude) into a pair of electrodes.

FIG. 4 is a table indicating reference difference signal magnitudes and phases for determining impedance, and thus quality, of pairs of electrodes.

FIG. 5A is a flow diagram for determining a quality of electrodes based on a difference signal generated using unbalanced injection currents that differ in both magnitude and phase.

FIG. 5B is a flow diagram for determining a quality of electrodes based on a difference signal generated using unbalanced injection currents that differ in phase only.

FIG. 5C is a flow diagram for determining a quality of electrodes based on a difference signal generated using unbalanced injection currents that differ in magnitude only.

FIGS. 6 and 7A-7B depict mechanisms for calculating reference difference signal magnitudes and phases for determining electrode impedances and qualities.

FIG. 8 depicts circuitry for generating a Wilson reference signal that is provided to the body node and to the V-lead differential amplifiers.

FIG. 9 is a diagram depicting an example ECG machine having electrode quality measurement functionality.

FIG. 10 is a flow diagram depicting a method of simultaneously determining impedances of a plurality of electrodes.

### DETAILED DESCRIPTION

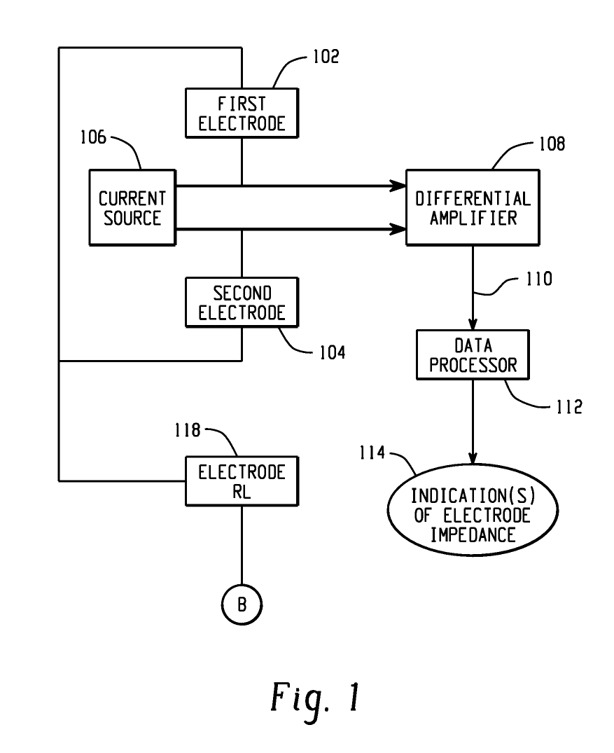

FIG. 1 is a block diagram depicting a system for simultaneously determining the quality of a plurality of electrodes (e.g., electrophysiological electrodes). As noted above, the quality of electrodes, such as ECG electrodes, can deteriorate over time. In order to maintain ECG measurement integrity, it is desirable for electrode impedance to be measured (e.g., periodically) in order to determine when the electrode is no longer a good transducer. When an electrode deteriorates beyond a certain point, that electrode should be replaced so that the low quality electrode does not interfere with signal acquisition. Such failures can be severely detrimental when they occur in scenarios that are time sensitive, such as emergency ECG measurement.

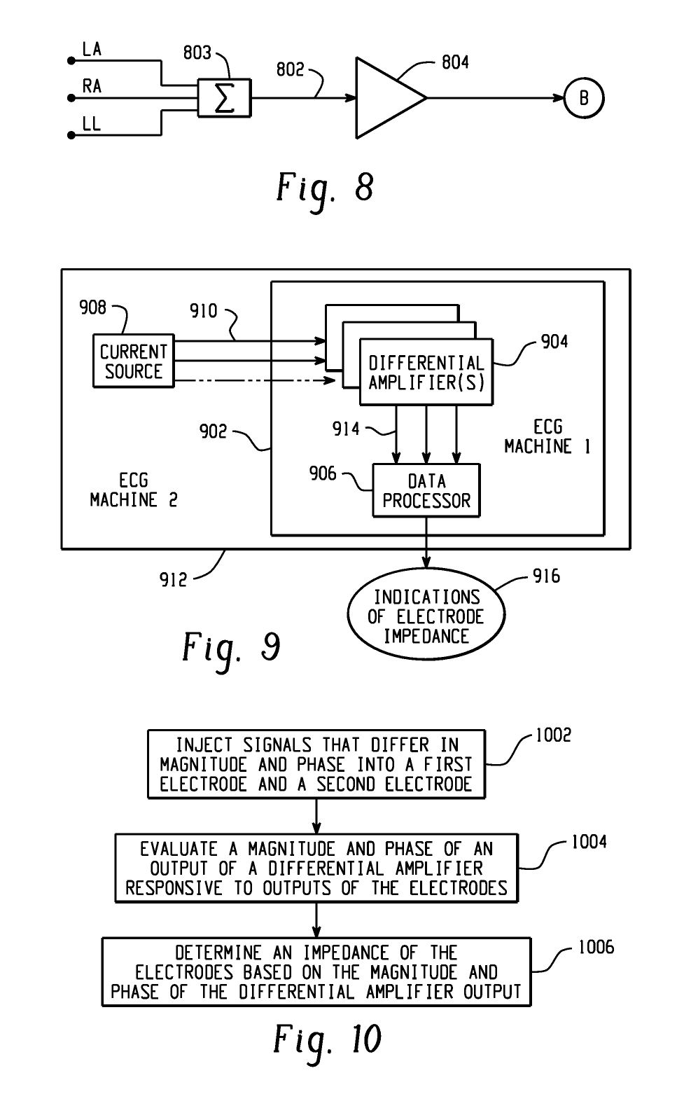

FIG. 1 depicts a system for measuring the quality of at least two electrodes at the same time to determine whether those electrodes are of sufficient quality, or if they should be replaced. In FIG. 1, the first end of a first electrode 102 is connected to the first output of a current source 106. The first end of a second electrode 104 is connected to the second output of the current source 106. The current source applies unbalanced differential alternating current (AC) currents signals (e.g., AC currents that differ in magnitude and phase) to its first and second outputs. A differential amplifier 108 also connects to and receives inputs from the first ends of the first electrode 102 and the second electrode 104. The differential amplifier 108 then generates an output difference signal at 110 that is indicative of the difference between the first ends of the electrodes 102, 104. That difference signal 110 is received by a data processor 112 that analyzes the difference signal 110 and determines a quality of both the first electrode 102 and the second electrode 104, such as based on a magnitude and phase of the difference signal 110. The second ends of electrode 102 and 104 are connected to the patient's body, to sense the desired electrophysiological signals (ECG, etc.). In addition, a right leg (RL) electrode is also connected to the patient body node. Any imbalance currents from current source 106 can thus be absorbed by the electrically neutral RL electrode. The second end of RL electrode 118 can be terminated in the neutral drive function shown in FIG. 8 on node B.

Based on that analysis, the data processor 112 outputs indications 114 of the quality of the first electrode 102 and the second electrode 104. Such indications 114 can take a variety of forms. In one example, the data processor 112 outputs estimated impedance values for each of the electrodes 102, 104, where impedances within different ranges indicate different electrode qualities. In another example, the data processor 112 outputs qualitative assessments of the electrodes 102, 104, such as “Good,” “Average,” and “Bad/Replace” based on the analysis of the difference signal 110. The data processor 112 can be configured to output the quality indications 114 to a variety of destinations, such as a computer-readable memory, a user interface of an ECG machine, one or more indicator lights of an ECG machine, or a graphical user interface of a computing device (e.g., a laptop, a tablet device) that is responsive to the system, such as via a wired or wireless connection.

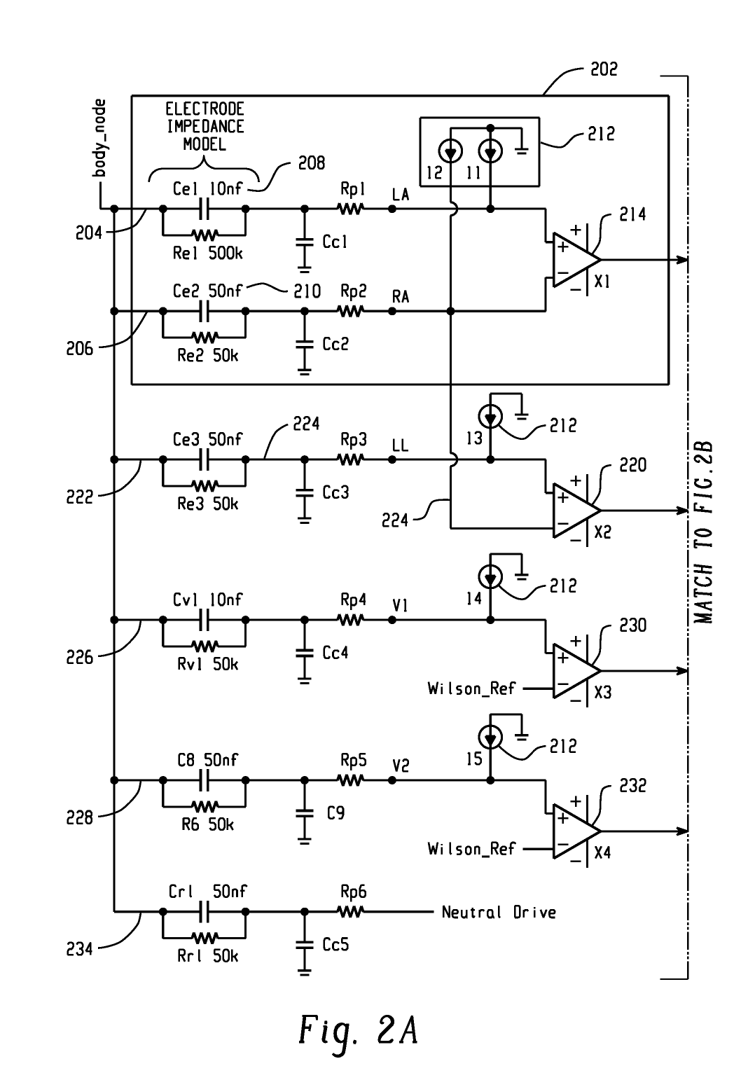

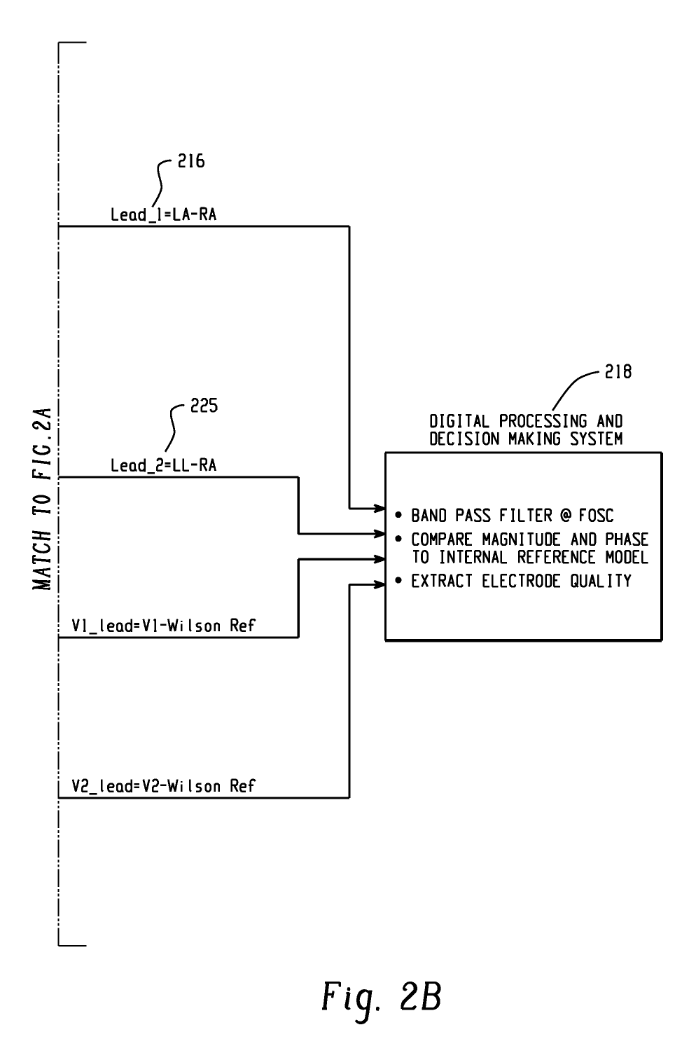

FIGS. 2A and 2B (hereinafter FIG. 2) are a diagram depicting exemplary components of a system for determining qualities of a plurality of electrodes simultaneously. Where the system of FIG. 1 determined the quality of two electrodes simultaneously, the system of FIG. 2 is capable of determining the quality of up to five electrodes at the same time. The circuitry depicted in box 202 roughly corresponds with the components labeled 102, 104, 106, 108, 110 in FIG. 1. Within box 202, a first branch 204 corresponds to a first electrode and a second branch 206 corresponds to a second electrode of an ECG system. Each branch 204, 206 includes a respective impedance 208, 210, modeled as a capacitance in parallel with a resistance. Over the lifespan of the electrodes 204, 206, the impedances 208, 210 are expected to change, the resistance increasing and the capacitance decreasing, as the quality levels of the electrodes deteriorate. Currents are injected into the electrodes 204, 206 by a current source 212, where the current source 212 injects a first current, I₁, into the first electrode 204 and a second current, I₂, into the second electrode 206. In one embodiment, those currents differ in both magnitude and phase. In certain other embodiments, those currents differ in magnitude or phase.

The circuitry within box 202 further includes a differential amplifier at 214, where the differential amplifier 214 is configured to receive outputs of both the first electrode 204 and the second electrode 206 when those electrodes are excited by the current source 212. The differential amplifier 214 generates a difference signal 216 that is indicative of the difference between the outputs of the first electrode 204 and the second electrode 206. That difference signal 216 is transmitted to a digital processing and decision making system 218 that determines the quality of the first electrode 204 and the second electrode 206 and outputs an indication of such.

In one embodiment, the differential amplifier 214 utilized to generate the difference signal 216 that is used for determining qualities of the electrodes 204, 206 is also utilized in normal device operation. For example, in an ECG machine implementation, the differential amplifier 214 depicted in FIG. 2 is used in normal ECG operation to detect a difference in potential between a left arm electrode (indicated as LA in FIG. 2) and a right arm electrode (indicated as RA in FIG. 2), a potential difference that is useful in generating a composite ECG signal that indicates a quality of a heart's function. Such reuse of the differential amplifier 214 in determining electrode 204, 206 qualities can limit an amount of additional hardware that needs to be incorporated into a system to enable electrode 204, 206 quality detection.

As depicted in FIG. 2, the system for detecting electrode quality can analyze more than two electrodes. Such operations can be in series with the measurement of the left arm 204 and right arm 206 electrodes or in parallel. Parallel operations can decrease the time necessary to evaluate all electrodes used in a system. Such speed can be highly beneficial in systems that utilize large numbers of electrodes, where a typical ECG machine operates using 10 electrodes positioned across a human being monitored. In the example of FIG. 2, a second differential amplifier 220 is configured to generate a second difference signal that indicates a difference of outputs of the left leg electrode 222 and the right arm electrode 206 of an ECG system. The second differential amplifier receives one input from the right arm electrode 206 via a connection indicated at 224, where that right arm electrode is excited by current I₂ from the current source 212. The left leg (third) electrode 222 is excited by another current I₃, which may also originate from the current source 212 and, in one example, is equal to current I₁. The difference signal 225 outputted from the second differential amplifier 220 is provided to the processing system 218 to determine an impedance, and thereby quality, of the left leg electrode 222 (that impedance being indicated by the impedance model at 224) and the right arm electrode 206.

A system may be expanded to determine qualities of a number of additional electrodes (e.g., electrodes 226, 228), as desired. In an ECG system, typically 10 electrodes are utilized, with six of those electrodes being V-lead electrodes. Two such V-lead electrodes are depicted at 226, 228. In the example of FIG. 2, V-lead electrode 226, 228 quality is measured in a similar fashion to the left arm 204, right arm 206, and left leg electrodes 222, where current I₄ is injected into the first V-lead 226, and current I₅ is injected into the second V-lead 228. In the V-lead examples, differential amplifiers 230, 232 receive one input from a respective electrode 226, 228 output and determine a difference relative to a reference voltage. In the example of FIG. 2, the reference voltage is a Wilson reference voltage, which is provided as the average of the left arm 204, right arm 206, and left leg 222 voltages (i.e., ⅓*(LA+RA+LL)), where circuitry for generating that Wilson reference signal is not shown in FIG. 2. FIG. 8 depicts circuitry for generating a Wilson reference signal that is provided to the V-lead differential amplifiers. The voltages from the left arm, right arm, and left leg electrodes are summed and divided by three at 803 to generate the Wilson reference voltage at 802. The Wilson reference voltage, 802 can also be applied to the input of the neutral drive feedback amplifier 804. The output of the neutral drive feedback amplifier 804, designated as node B in FIGS. 1 and 8, is used to drive the second end of the RL electrode in FIG. 1 to absorb any current source imbalances from the current source 106 in FIG. 1.

In one embodiment, the sampling period of the measured electrode impedance can be relatively low, not needing to be updated more than about every 30 seconds. This enables use of AC current stimulus signal magnitudes that are small, below the input referred noise level in an ECG system. Measurement signals can then be recovered via averaging, easing possible burdens of post-filtering of the ECG signal to remove the AC current “carrier wave” stimulus signals.

In certain embodiments of the disclosure, characteristics of the currents injected into the electrodes, such as by current source 212, aid in the ability of the processing system 218 to determine electrode quality. In one embodiment, the system utilizes unbalanced AC current sources injected into each electrode. The unbalanced current sources can aid in measurement of electrode impedances that change in a common mode fashion by maintaining a differential output signal even when the two corresponding electrode impedances change identically or substantially identically. In certain embodiments, if common currents were injected into electrode pairs (e.g., 204, 206), the differential amplifier (e.g., 214) would reject common mode impedance changes (and therefore common mode voltages) seen at its inputs. In one embodiment, the AC current imbalance (e.g., between I₁ and I₂) is at a 2:1 ratio in magnitude (e.g. I₁=10 nA, I₂=5 nA), where the currents are offset in phase (e.g., by 180 degrees). The currents can be designed and arranged such that there is little or no net sum current flow into the body node and neutral drive electrode 234 if desired. Currents for the V-lead electrodes 226, 228 can be similarly designed, as equal in magnitude and opposite in phase, such that the net sum of the currents into the body node 234 is zero.

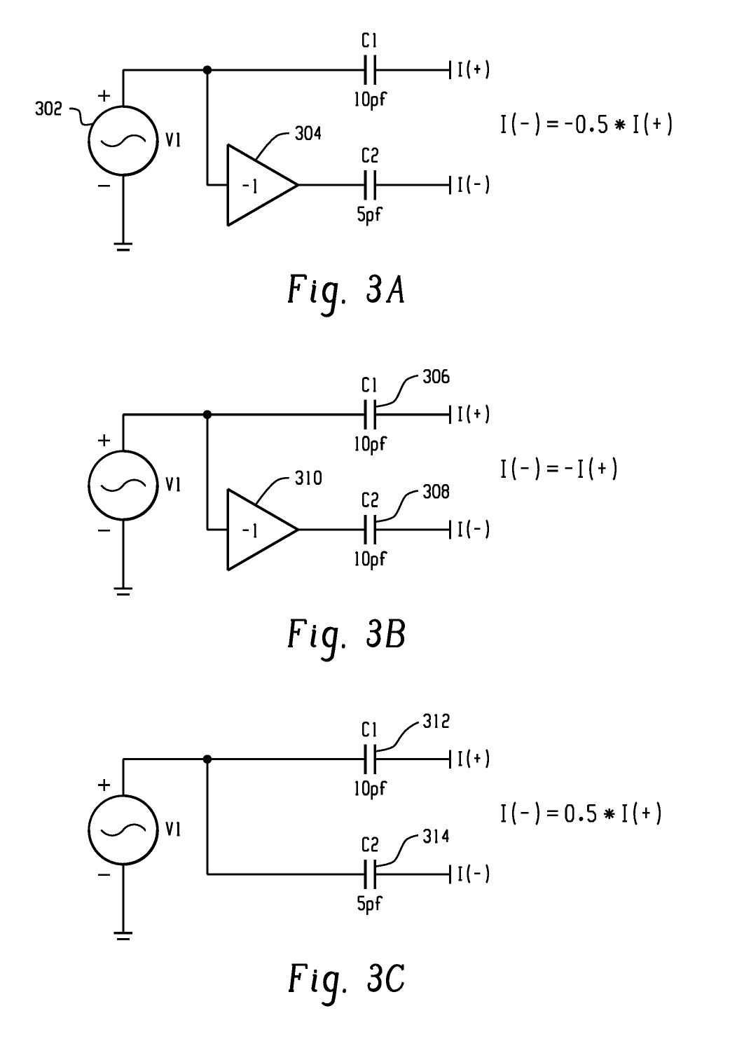

FIGS. 3A-3C depict example current sources for injecting unbalanced currents into a pair of electrodes. FIG. 3A is a diagram depicting a first example current source for injecting currents that are unbalanced in both magnitude and phase into a pair of electrodes (e.g., I₁ and I₂ in FIG. 2). A sinusoidal voltage source 302 generates a voltage V₁ at a frequency of F₀. Magnitudes of currents outputted from the two outputs I₊ and I₋ are controlled by capacitors C₁ and C₂ positioned on respective branches of the current source. Those capacitors are selected to control magnitudes of currents outputted from the current source of FIG. 3. In the example depicted in FIG. 3, the magnitude of I₋ is 0.5 that of I₊. The phase of the current outputted from the I₋ output is lagged by 180 degrees via an inverter 304 positioned on the I₋ branch.

FIG. 3B depicts another example that produces currents that are unbalanced in phase only for injection into a pair of electrodes. In contrast to FIG. 3A, the example of FIG. 3B includes two identical (or similarly) sized capacitors 306, 308 on its output branches, resulting in injection currents having the same (or similar) magnitudes, with opposite phases based on the positioning of inverter 310 on one of the output branches. FIG. 3C depicts a further example that produces currents that are unbalanced in magnitude only for injection into a pair of electrodes. In contrast to FIG. 3A, the example of FIG. 3C does not include an inverter on either of the output branches. The two differently sized capacitors 312, 314 on the respective output branches result in currents that are unbalanced in magnitude being outputted from the current source of FIG. 3C.

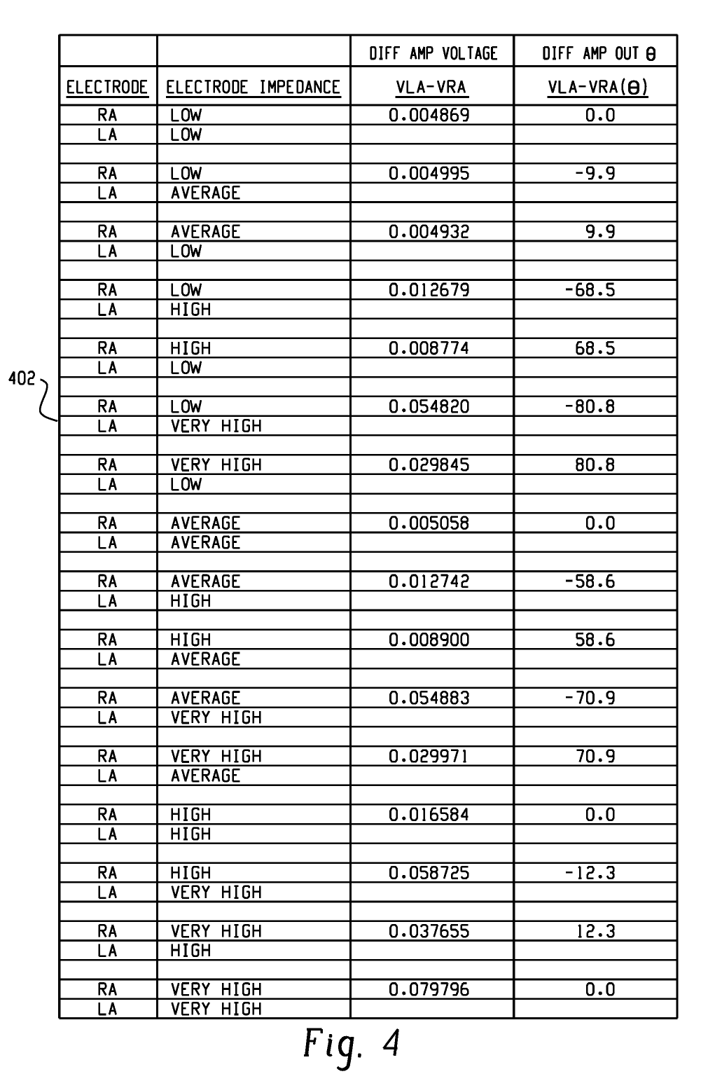

As described above, each electrode of a pair of electrodes connected to a differential amplifier is injected with one of a pair of unbalanced currents. The resulting difference signal produced by the differential amplifier (following filtering, such as at the AC input current frequency F₀) will have a magnitude and phase, where that magnitude and phase is indicative of the quality of both of the electrodes connected to the differential amplifier. FIG. 4 is a table illustrating representative reference difference signal magnitudes (V_{LA}−V_{RA}) and phases (V_{LA}−V_{RA} (θ)) for determining impedance, and thus quality, of pairs of electrodes. In the example of FIG. 4, unbalanced currents are injected into the right arm electrode and left arm electrode, and the magnitude and phase of a resulting difference signal is observed. The quality of the two electrodes can then be determined based on that magnitude and phase. It is noted that the threshold values depicted in FIG. 4 are exemplary in nature and may be changed (e.g., according to the magnitudes and phases of the input currents, the desired electrode quality cutoffs, etc.).

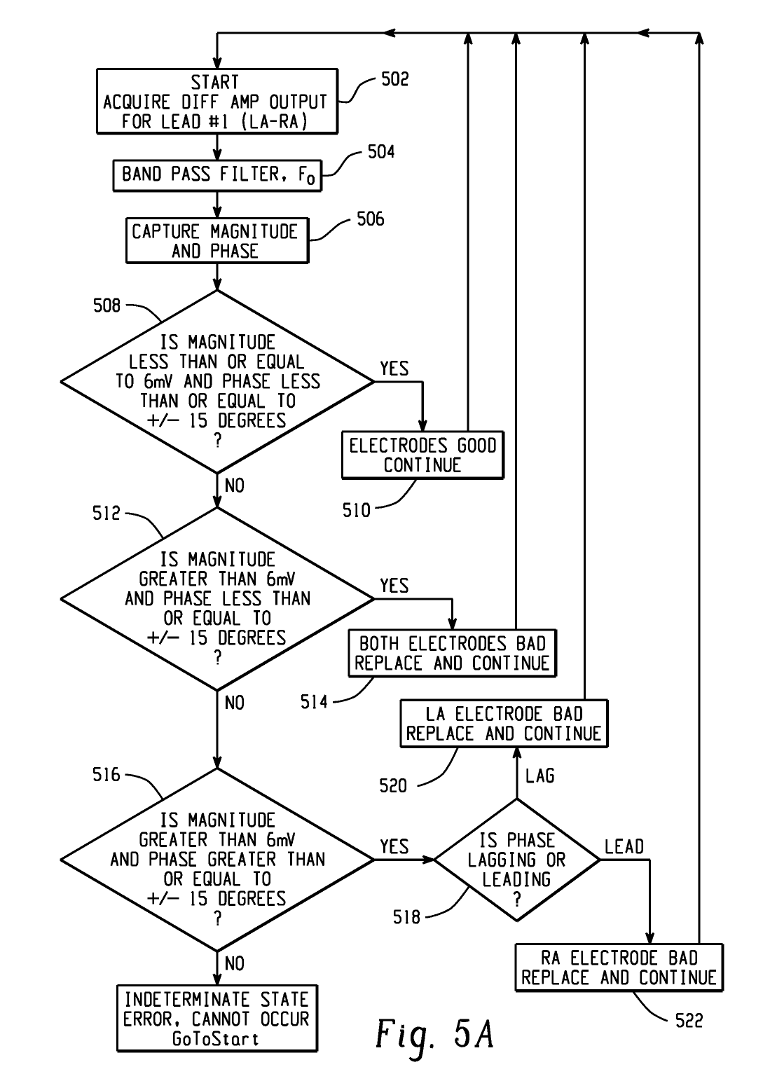

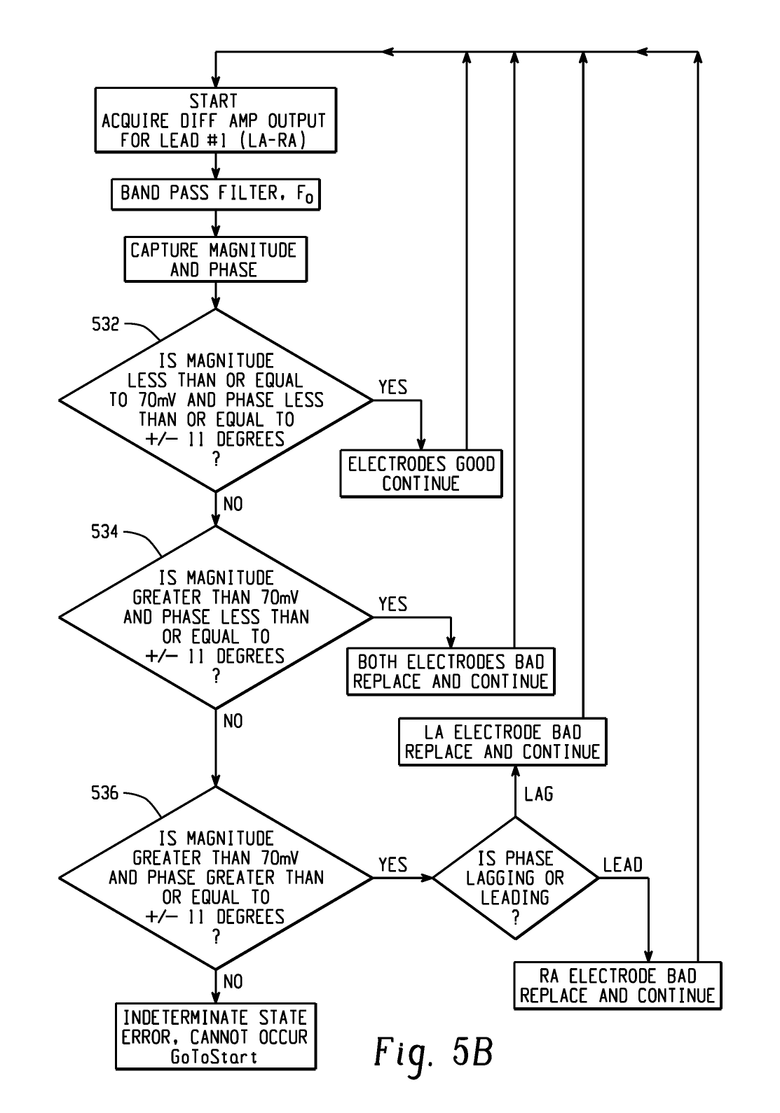

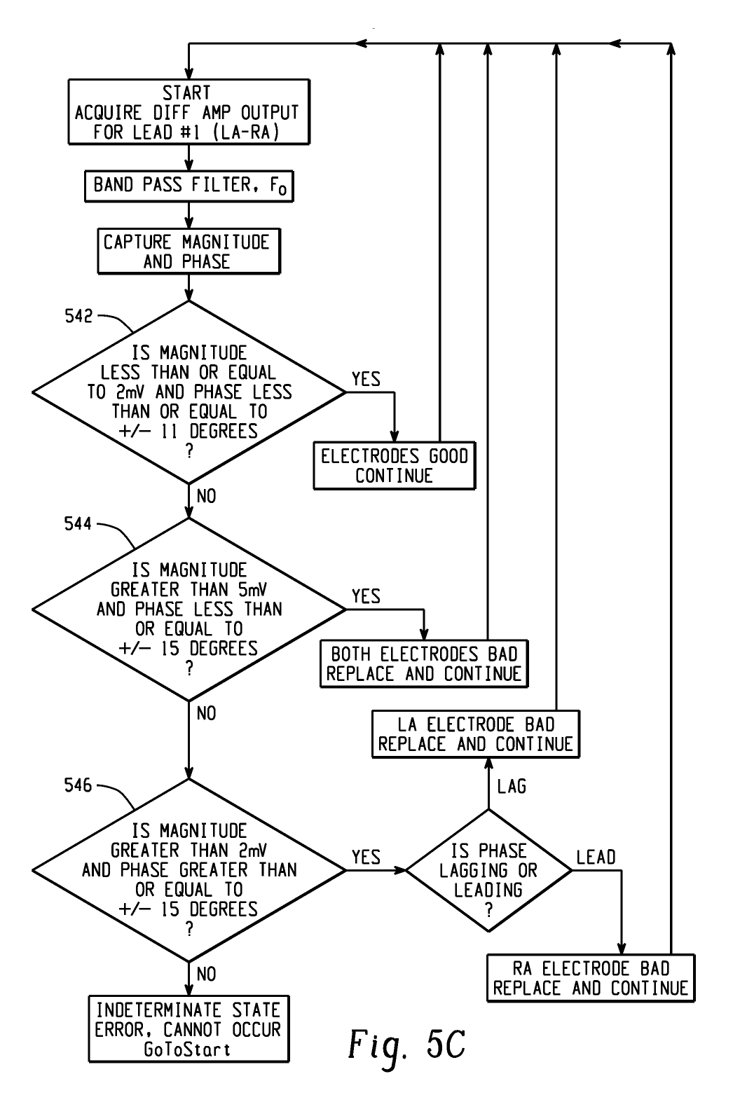

As described with reference to FIG. 4, FIG. 5A details an algorithm for determining electrode quality based on a difference signal. At 502, a difference signal is acquired from the output of a differential amplifier based on inputs from two electrodes (e.g., electrode LA and electrode RA). At 504, that difference signal is band pass filtered at the frequency, F₀, of the current source. The magnitude and phase of the resulting signal is captured at 506. A series of comparisons are then performed against a series of thresholds to determine the quality of the two electrodes. (Note, the thresholds in the algorithm can be varied, as described further herein, such as based on characteristics of the currents injected into the electrodes.) At 508, a determination is made as to whether the magnitude of the difference signal is less than or equal to 6 mV and the absolute value of the phase is less than or equal to 15 degrees. If so, a determination is made at 510 that both electrodes are in good condition. If not, at 512, a determination is made as to whether the magnitude of the difference signal is greater than 6 mV and the absolute value of the phase is less than or equal to 15 degrees. If so, a determination is made at 514 that both electrodes are in bad condition and should be replaced. If not, at 516, a determination is made as to whether the magnitude of the difference signal is greater than 6 mV and the absolute value of the phase is greater than 15 degrees. If so, and the phase is lagging at 518 (i.e., negative), then the left arm electrode is deemed bad at 520 and should be replaced, while the right arm electrode is deemed of sufficient quality. In contrast, if the phase is leading at 518, then the right arm electrode is deemed bad at 522 and should be replaced, while the left arm electrode is deemed of sufficient quality. It is further possible to measure electrode impedance degradation over time by repeating these measurements utilizing different decision thresholds of varying sensitivities.

As noted above with reference to FIGS. 3B and 3C, currents can be injected into electrodes that differ in phase only (FIG. 3B) or magnitude only (FIG. 3C). Algorithms for evaluating electrode qualities based on those types of injected currents are depicted in FIGS. 5B and 5C, respectively. In FIG. 5B, corresponding to injected currents that differ in phase but not magnitude, the threshold voltages and phases in the algorithm inquiries are adjusted (i.e., ≦70 mV and ≦±11 degrees at 532, >70 mV and ≦±11 degrees at 534, and >70 mV and ≦±11 degrees at 536. In the example of FIG. 5C, corresponding to injected currents that differ in magnitude but not phase, the threshold voltages and phases in the algorithm inquiries are adjusted (i.e., ≦2 mV and ≦±11 degrees at 542, >5 mV and ≦±15 degrees at 544, and >2 mV and ≦±15 degrees at 546).

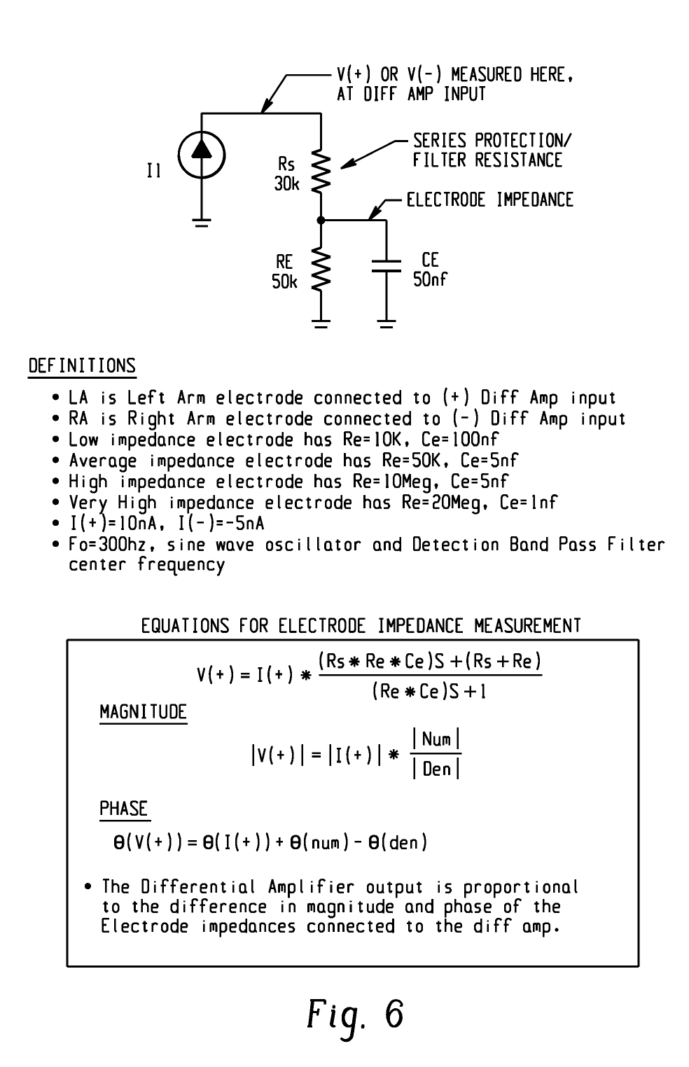

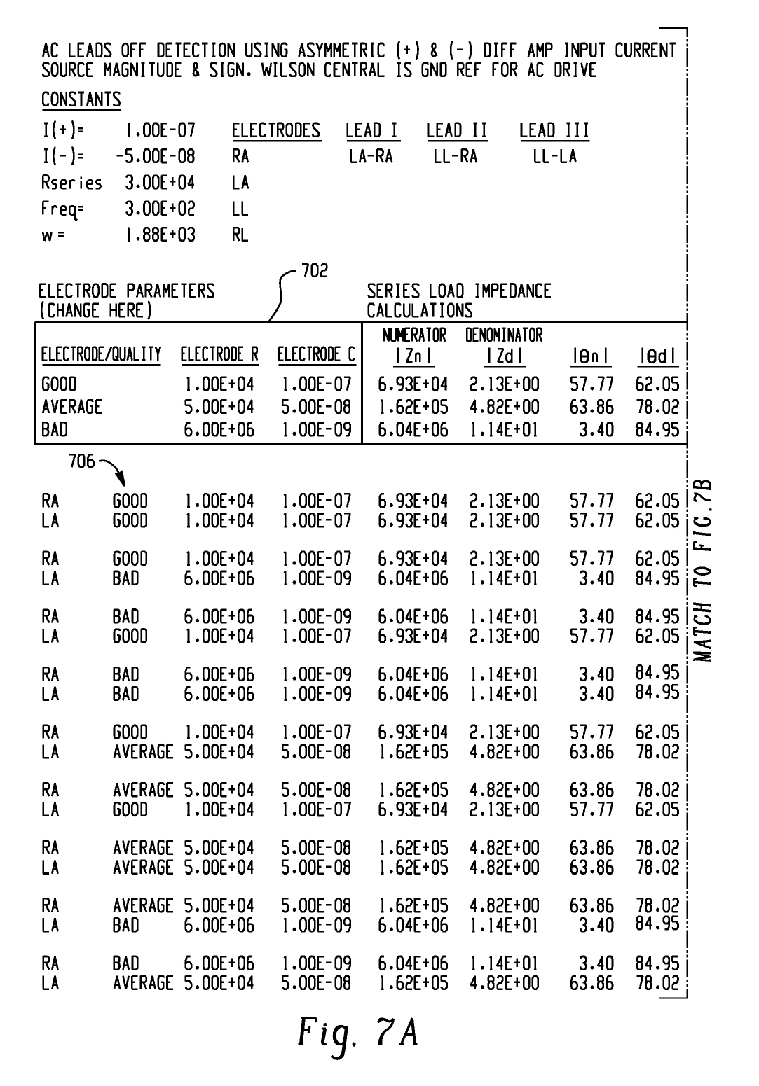

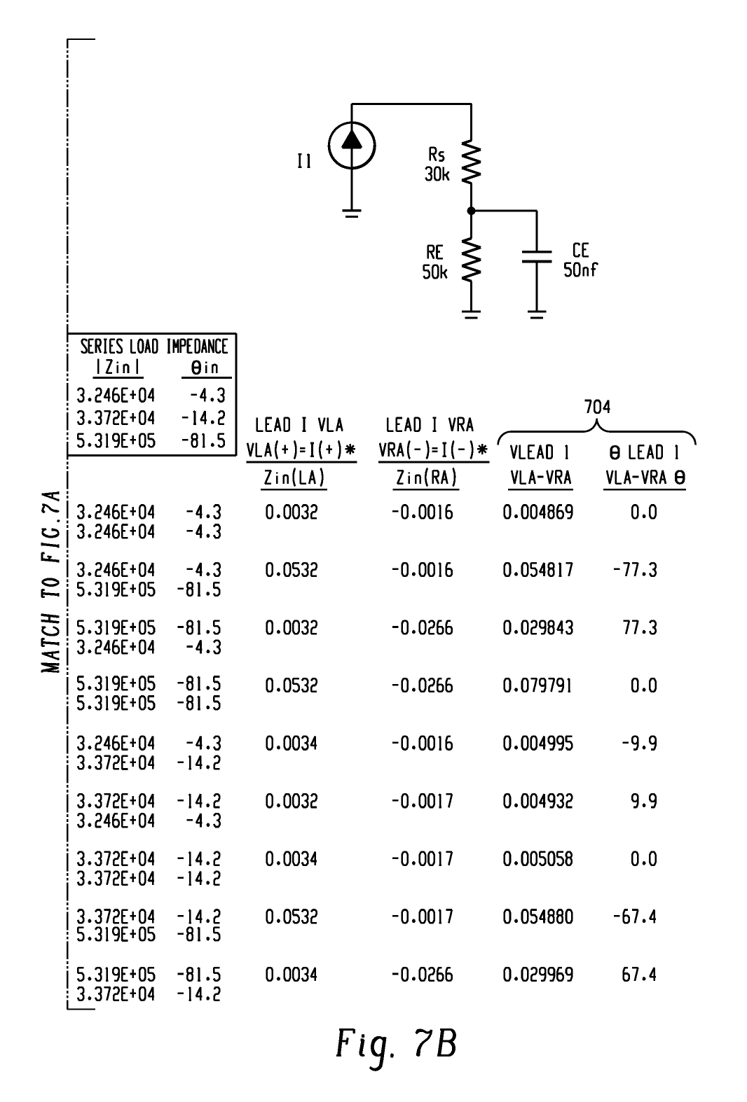

The reference magnitudes and phases depicted in FIGS. 4 and 5A-C can be determined in a variety of ways. FIGS. 6 and 7A-7B (hereinafter FIG. 7) depict mechanisms for calculating reference difference signal magnitudes and phases for determining electrode impedances and qualities based on the injection currents of FIG. 5A that vary in both magnitude and phase. FIG. 6 illustrates a model of the electrode to be evaluated, where voltage caused by an injected current I₁ is measured at the V₊ or V₋ differential amplifier input. A series/protection/filter resistance is represented by a constant R_{S}, and an electrode impedance is represented by a parallel resistance R_{E} and capacitance C_{E}. To determine reference difference signal magnitudes and phases (e.g., for use in the algorithms of FIG. 4 or 5) systems of equations representing the difference between the voltage generated at V₊/V₋ for two such electrodes are solved, using different values for Rₑ and Cₑ to represent electrodes of different qualities. In the example of FIG. 6, a low impedance (and thus high quality electrode) is represented by a resistance of 10 K Ohms and a capacitance of 100 nanofarads. An average impedance (and thus an average quality electrode) is represented by a resistance of 50 K Ohms and a capacitance of 50 nanofarads. A high impedance (and thus an electrode in a low quality state) is represented by a resistance of 10 M Ohms and a capacitance of 5 nanofarads. The equation solving further utilizes the characteristics of the unbalanced current sources, presented in FIG. 6 as 10 nA and −5 nA (i.e., 5 nA at a phase shift of 180 degrees) at 300 Hz.

The table of FIG. 7 depicts results from example equation solving to identify reference difference signal magnitudes and phases. Impedance parameters of what is considered a good, average, or bad quality electrode are inputted at 702 and a table of reference values is generated at the bottom of FIG. 7. The right two columns of the bottom table of FIG. 7 indicate reference difference signal magnitudes and phases at 704. Each reference difference signal magnitude/phase pair corresponds with a quality of both the left arm and right arm electrodes being evaluated, with that quality being indicated in the second column of the table at 706. In one embodiment, a magnitude and phase of a difference signal acquired from a differential amplifier is compared to the reference magnitude/phase thresholds to make a determination of electrode quality, as is shown in the flowchart of FIG. 5.

FIG. 9 is a diagram depicting an example ECG machine having electrode quality measurement functionality. FIG. 9 indicates two example boundaries for an ECG machine. A first example ECG machine 902 includes integrated differential amplifiers 904 and a data processor 906. As mentioned above, functionality for determining electrode quality can utilize differential amplifiers 904 that are used in normal ECG measurement mode to capture voltages across different terminals on a human body. The first ECG machine 902 utilizes an external current source 908 for injecting currents into two or more electrodes 910. In a second example of FIG. 9, a second ECG machine configuration 912 is depicted that includes the current source functionality 908. In either ECG machine configuration 902, 912, the two or more electrodes 910 are connected to the external/integrated current source 908, where unbalanced AC currents are applied. One or more difference signals 914 are generated by the differential amplifiers 904, as described herein, and the difference signals are analyzed by a data processor 906 to generate indications of electrode impedance/quality at 916.

FIG. 10 is a flow diagram depicting a method of simultaneously determining impedances of a plurality of electrodes. At 1002, signals are injected into a first electrode and a second electrode, the injected signals differing in at least one of magnitude and phase. A magnitude and phase of an output of a differential amplifier are evaluated at 1004, where the differential amplifier is responsive to outputs of the first electrode and the second electrode. At 1006, an impedance of the first electrode and an impedance of the second electrode are determined based on the magnitude and the phase of the differential amplifier output.

Examples have been used herein to describe exemplary aspects of the current subject matter, but the scope of this disclosure encompasses other examples and should not be limited thereto. For example, the frequency of the currents injected into the electrodes can be varied to facilitate measurement of various aspects of electrode impedance. In the examples described above, a mid-range frequency of 300 Hz for injected currents was utilized because that frequency facilitates measurement of both the resistive and capacitive portions of an electrode's impedance. In some implementations, separate measurement of one or both of those portions is desirable.

As described above, an electrode can be modeled as a parallel R/C circuit. Such a circuit has a corner frequency equal to 1/(2*Pi*R*C). The impedance of the electrode, Z_{elect}, is approximately constant for input currents having a frequency below this corner frequency with a value of Z_{elect}=R. Selecting an input current frequency near the corner frequency will facilitate measurement of a combination of the resistive and capacitive components of the impedance. Above the corner frequency, the impedance of the electrode will decrease with increasing frequency, and have a value of approximately Z_{elect}=(1/(2*Pi*f*C)), where f is the injected current frequency. Thus, input currents having frequencies below the corner frequency will measure primarily the resistive component of the electrode impedance, and input currents above the corner frequency will measure primarily the capacitive component of the electrode.

In certain embodiments, one of the resistive and capacitive portions of the electrode impedance is more important to the electrode application. For example, where electrodes are used to sample low frequency voltage changes, such as changes caused by respiration, the electrode signal capture ability is more sensitive to capacitance changes to the electrode. Where electrodes are used to sample high frequency voltage changes, such as those caused by a heartbeat, resistance changes have a more substantial effect on the quality of electrode performance. Thus, where only one of the resistive or capacitive portions of the electrode impedance is important, a corresponding input current frequency can be selected. That is, a higher precision capacitance evaluation can be performed by injecting a high frequency current (e.g., 1000 Hz) into the electrodes. Conversely, a higher precision resistance evaluation can be performed by injecting a low frequency current (e.g., 3 Hz) into the electrodes. Reference magnitude/phase pairs as described herein would be adjusted accordingly.

In a further embodiment, a system can be configured to utilize input currents that are a composite of two frequencies I_{HF} and I_{LF} (e.g., I_{HF}=1000 Hz, I_{LF}=3 Hz). The difference signal from a differential amplifier can be band pass filtered across two different branches (i.e., one branch at 1000 Hz and a second branch at 3 Hz) to extract a magnitude and phase for each component frequency. The high frequency magnitude/phase values can be used to evaluate the capacitance of the electrode, while the low frequency magnitude/phase values can be used to evaluate the resistance of the electrode, in parallel. In certain embodiments, such a composite frequency configuration can be used to simultaneously acquire higher resolution characterizations of both the capacitive and resistive components of impedances for multiple electrodes at the same time, with limited additional hardware over other embodiments described herein.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A method of simultaneously determining impedances of a plurality of electrophysiological electrodes, comprising:
injecting signals into a first electrophysiological electrode and a second electrophysiological electrode, the injected signals differing in at least one of magnitude and phase;
evaluating a magnitude and phase of an output of a differential amplifier, wherein the differential amplifier is responsive to outputs of the first electrophysiological electrode and the second electrophysiological electrode;
wherein an impedance of the first electrophysiological electrode and an impedance of the second electrophysiological electrode are determined based on the magnitude and the phase of the differential amplifier output.

2. The method of claim 1, further comprising:
outputting an indication of whether the impedance of either or both of the first electrophysiological electrode and the second electrophysiological electrode indicate an electrode is in a low quality state.

3. The method of claim 2, wherein electrophysiological electrodes are components of an electrocardiogram (ECG) machine, and wherein the indication indicates that an electrophysiological electrode in a low quality state is to be replaced.

4. The method of claim 1, wherein the impedance of the first electrophysiological electrode and the impedance of the second electrophysiological electrode are determined by:
determining that both the first electrophysiological electrode and the second electrophysiological electrode have impedances that indicate that those electrodes are not in a low quality state when the magnitude of the differential amplifier output and an absolute value of the phase of the differential amplifier output are below predetermined thresholds.

5. The method of claim 1, wherein the impedance of the first electrophysiological electrode and the impedance of the second electrophysiological electrode are determined by:
determining that both the first electrophysiological electrode and the second electrophysiological electrode have impedances that indicate that those electrodes are in a low quality state when the magnitude of the differential amplifier output exceeds a magnitude threshold and an absolute value of the phase of the differential amplifier output is below a phase threshold.

6. The method of claim 1, wherein the impedance of the first electrophysiological electrode and the impedance of the second electrophysiological electrode are determined by:
determining that exactly one of the first electrophysiological electrode and the second electrophysiological electrode has an impedance that indicates that that one of those electrodes is in a low quality state when the magnitude of the differential amplifier output exceeds a magnitude threshold and an absolute value of the phase of the differential amplifier output exceeds a phase threshold; and
determining which of the first electrophysiological electrode and the second electrophysiological electrode has the impedance that indicates that that electrode is in a low quality state based on whether the phase of the differential amplifier output is positive or negative.

7. The method of claim 1, wherein the impedance of the first electrophysiological electrode and the impedance of the second electrophysiological electrode are determined by comparing the magnitude and phase of the output of the differential amplifier to a plurality of reference magnitude/phase pairs and selecting a closest reference magnitude/phase pair;
wherein each reference magnitude/phase pair indicates an impedance of the first electrophysiological electrode and the second electrophysiological electrode.

8. The method of claim 1, wherein the signals injected into the first electrophysiological electrode and the second electrophysiological electrode are comprised of a plurality of frequencies.

9. The method of claim 1, wherein the signals injected into the first electrophysiological electrode and the second electrode are composite signals having a high frequency component and a low frequency component.

10. The method of claim 9, wherein resistances of the first electrophysiological electrode and the second electrophysiological electrode are determined based on the low frequency component of the injected signals; and
wherein capacitances of the first electrophysiological electrode and the second electrophysiological electrode are determined based on the high frequency component of the injected signals.

11. The method of claim 1, wherein the signals injected into the first electrophysiological electrode and the second electrophysiological electrode are current signals generated by a current source.

12. The method of claim 1, further comprising:
injecting a signal into a third electrophysiological electrode;
evaluating a magnitude and phase of an output of a second differential amplifier, wherein the second differential amplifier is responsive to an output of the third electrophysiological electrode;
wherein an impedance of the third electrophysiological electrode is determined based on the magnitude and the phase of the second differential amplifier output.

13. The method of claim 12, wherein the second differential amplifier is further responsive to the output of the second electrophysiological electrode.

14. The method of claim 12, wherein the second differential amplifier is further responsive to a reference voltage.

15. The method of claim 14, wherein the reference voltage is a Wilson reference voltage.

16. The method of claim 12, wherein impedances of the first electrophysiological electrode, the second electrophysiological electrode, and the third electrophysiological electrode are determined at substantially the same time.

17. The method of claim 1, wherein the injected signals differ in both magnitude and phase.

18. The method of claim 1, wherein the injected signals differ in magnitude but not phase.

19. The method of claim 1, wherein the injected signals differ in phase but not magnitude.

20. A system for simultaneously determining impedances of a plurality of electrophysiological electrodes, comprising:
a current source configured to inject signals into a first electrophysiological electrode and a second electrophysiological electrode, the injected signals differing in at least one of magnitude and phase;
a differential amplifier configured to receive an output of the first electrophysiological electrode and an output of the second electrophysiological electrode, the differential amplifier being further configured to output a difference signal;
a data processor configured to determine an impedance of the first electrophysiological electrode and an impedance of the second electrophysiological electrode based on a magnitude and phase of the difference signal.

21. The system of claim 20, wherein the first electrophysiological electrode and the second electrophysiological electrode are electrophysiological electrodes of an electrocardiogram machine.

22. The system of claim 21, wherein the differential amplifier is further configured to be utilized in an electrocardiogram measurement operation in addition to utilization in determining impedances of the first electrophysiological electrode and the second electrophysiological electrode.

23. The system of claim 20, wherein the current source comprises:
a voltage source;
a first branch comprising a first capacitor having a first capacitance;
a second branch comprising an inverter and a second capacitor having a second capacitance that differs from the first capacitance.

24. The system of claim 20, wherein the system comprises an electrocardiogram machine.

25. An electrocardiogram machine configured to determine impedances of a plurality of electrophysiological electrodes connected to the electrocardiogram machine, signals that differ in at least one of magnitude and phase being injected into a first electrophysiological electrode and a second electrophysiological electrode, the electrocardiogram machine comprising:
a differential amplifier configured to receive an output of the first electrophysiological electrode and an output of the second electrophysiological electrode, the differential amplifier being further configured to output a difference signal;
a data processor configured to determine an impedance of the first electrophysiological electrode and an impedance of the second electrophysiological electrode based on a magnitude and phase of the difference signal.

26. The electrocardiogram machine of claim 25, further comprising:
a current source for generating the signals, which differ in magnitude and phase, injected into the first electrophysiological electrode and the second electrophysiological electrode.
